(19) United States
(12) Patent Application Publication
(10) Pub. No.: US 2017/0265732 A1
(43) Pub. Date: Sep. 21, 2017

(54) LIGHTING ASSEMBLY FOR CAMERA MODULE OF AN ENDOSCOPE

(71) Applicant: CHIYI TECHNOLOGY CO., LTD., NEW TAIPEI CITY (TW)

(72) Inventor: YEN TSUNG LIN, NEW TAIPEI CITY (TW)

(21) Appl. No.: 15/610,935

(22) Filed: Jun. 1, 2017

Related U.S. Application Data
(63) Continuation-in-part of application No. 15/332,019, filed on Oct. 24, 2016.

(30) Foreign Application Priority Data
Mar. 11, 2016 (TW) 105203353
Apr. 26, 2017 (TW) 106205832

(51) Int. Cl. A61B 1/06; H04N 5/225

(52) U.S. Cl. CPC A61B 1/0607; H04N 5/2256; H04N 5/2253; A61B 1/0684; A61B 1/0676; H04N 2005/2255; A61B 1/00105

(57) ABSTRACT

The present invention is aimed to design an improved lighting assembly for camera module of an endoscope. The camera module is received in a cylinder body, which is connected with the tail end of the tube of the endoscope. The character of this invention is to provide a secondary lighting apparatus between the cylinder body and the tube. The secondary lighting apparatus includes several LEDs being mounted inside annular-arrayed and covered by a transparent ring. The annular-arrayed LEDs can emit light through the transparent ring with 360 degrees lighting outwards that provides a perfect lighting effect for capturing the inner images of patients and will facilitate the judgement of users or doctors.

### CROSS-REFERENCE TO RELATED APPLICATIONS

This application claims the benefit of U.S. patent application Ser. No. 15/332,019, filed on Oct. 24, 2016, currently pending, which claims the priority of Taiwan Utility Application No. 105203353, filed on Mar. 11, 2016, now Utility Patent No. M524474, and the entire contents of which are incorporated herein by reference in their entirety. This Application also claims the priority of Taiwan Utility Application No. 106205832, filed on Apr. 26, 2017, the entire contents of which are incorporated herein by reference in their entirety.

### BACKGROUND OF THE INVENTION

It is known that the prior lighting source of an endoscope is used of a glass-fiber to transport the light made from a lighting source mounted in the control handle. Since the glass-fiber is possibly broken, it is troublesome when producing, and is more harmful and dangerous if the glass-fiber is broken during proceeding a medical surgery. In late years, it had developed a new lighting structure for the endoscope. As shown in FIG. 1, this known structure includes a cylinder body (1), which has provided with several lighting sources (11), such as LEDS, at front surface around the camera module (12). In use, the light of the lighting of the sources (11) is directly emitting forward and will be reflected because the cavity of human has glossy surface and is full of liquid that will occur a lighting spot on the signal of the image captured by the camera. It leads a blind area and increases risk of surgery. Moreover, since all lighting sources are mounted at the front portion of the cylinder body, the produced high heat may cause another danger that hurts the patient. Hence, the known designs are not utilized and should be improved. The present applicant had filed a U.S. patent application with Ser. No. 15/332,019 on Oct. 24, 2016, in which the cylinder body has provided with two side openings covered by related transparent caps. Several lighting sources are mounted in the cylinder body related to the position of the openings that provides side lights to increase whole lighting effect in the working cavity and will facilitate to obtain clear image captured from camera module. Since the light is emitted only through two sides of the cylinder, the light shadow will be still existed, that may still lead mistakes by the doctor possibly and this could be improved.

### SUMMARY OF THE INVENTION

The present invention is aimed to design an improved lighting assembly for camera module of an endoscope which can overcome the drawbacks mentioned in prior art. The camera module is received in a cylinder body, which is connected with the tail end of the tube of the endoscope. The character of this invention is to provide a secondary lighting apparatus between the cylinder body and the tube. The secondary lighting apparatus includes several LEDs being mounted inside annularly and covered by a transparent ring. In use, the annular-arrayed LEDs can emit light through the transparent ring with 360 degrees lighting outwards that provides a perfect lighting effect for capturing the inner images of patients and will facilitate the judgement of users or doctors. Now, accompanying with drawings, the character and structure of the present invention will be disclosed as following.

### BRIEF DESCRIPTION OF THE DRAWINGS

FIG. 1 is a perspective view showing the lighting assembly, the camera module, and the cylinder body of a conventional endoscope.

FIG. 2 is a perspective view showing the improved lighting assembly according to the present invention.

FIG. 3 is an exploded perspective view of FIG. 2.

FIG. 4 is a vertical cross-sectional plan view of FIG. 2.

FIG. 5 is a horizontal cross-sectional plan view of FIG. 2.

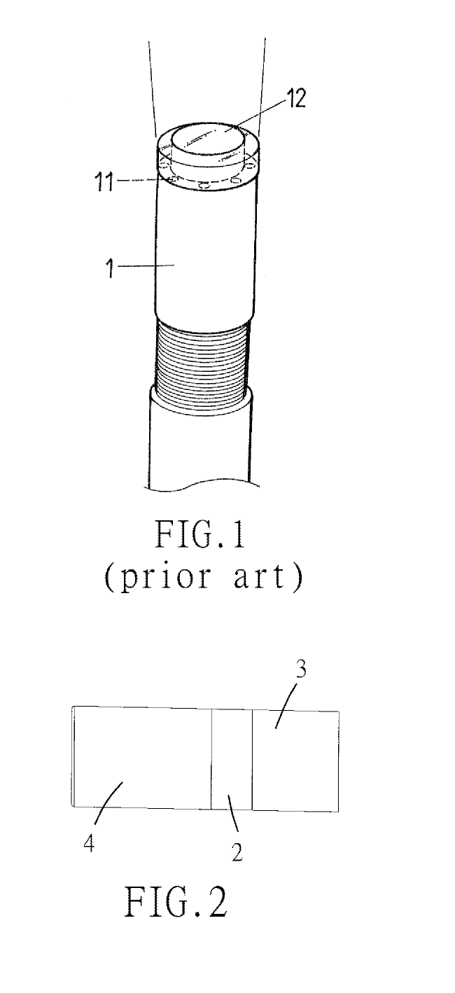

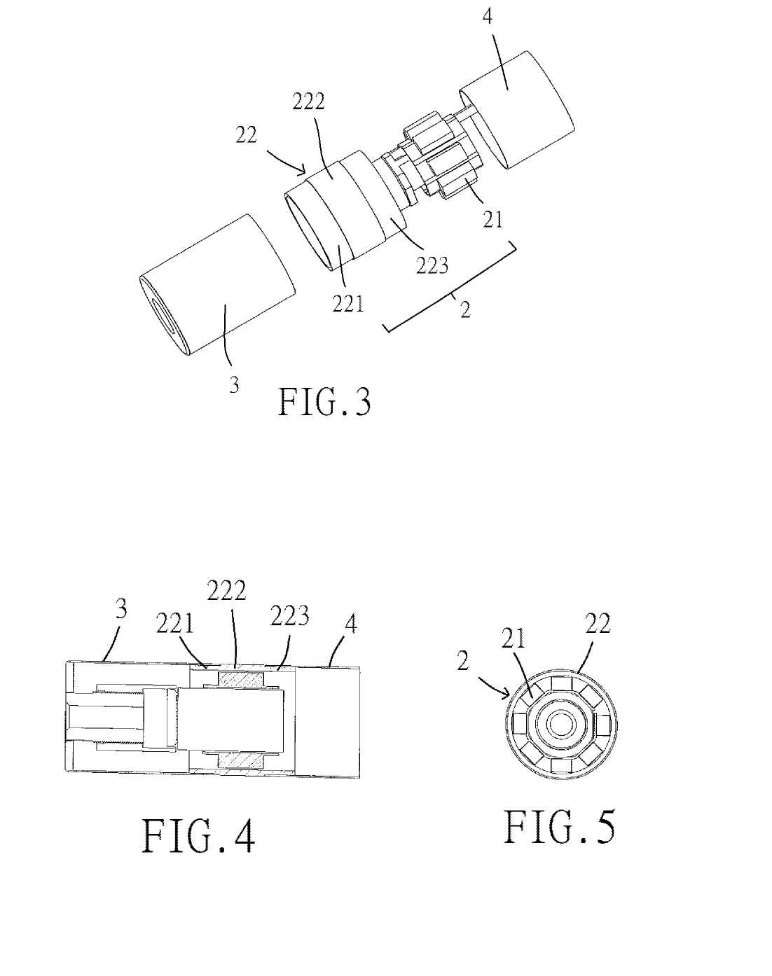

### DETAILED DESCRIPTION OF THE PREFERRED EMBODIMENT

Referring to FIG. 2, the present invention relates to an improved lighting assembly for camera module of an endoscope. The camera module is received in the cylinder body (3) at the tail end of the tube (4) of an endoscope. The character of the invention is to provide a secondary lighting apparatus (2) between the cylinder body (3) and the tube (4). The secondary lighting apparatus (2) includes several LEDs (21) being annularly arrayed inside, which are covered by a transparent ring (22). The transparent ring (22) is composed by an upper fringe (221), a middle fringe (222), and a lower fringe (223). The outer diameter of the middle fringe (222) is equal to the outer diameter of the tube (4) and the cylinder body (3). The outer diameter of the upper fringe (221) is equal to the inner diameter of the cylinder body (3). The outer diameter of the lower fringe (223) is equal to the inner diameter of the tube (4). Hence, the secondary lighting apparatus (2) can connected tightly with the cylinder (3) and the tube (4). In use, the annular-arrayed LEDs (21) can emit the light through the transparent middle fringe (222) and 360 degrees outwards. It is to be understood that the present invention can provide a perfect lighting effect for the endoscope to capture the inner images of patients and will facilitate the judgement for users or doctors to prevent from mistakes because of the unclear images.

Moreover, the secondary lighting apparatus is capable of providing with different color with related control means. By use of the different color of lighting, it is possible for doctors to find the cause of disease. Above all, the present obtains utility in no doubt. It is obviously utilized and effective for patient during medical procedures. Thus, it is to be understood that the present is allowable.

What is claimed is:

1. An improved lighting assembly for camera module of an endoscope including a tube connecting the tail end and a cylinder body receiving with a camera module, and the characteristic is in that:
a secondary lighting apparatus being provided between the cylinder body and the tube, which comprises several LEDs being annularly arrayed inside and covered by a transparent ring.

2. The improved lighting assembly for camera module of an endoscope as claimed in claim 1, wherein the LEDs are provided with different colors.

3. The improved lighting assembly for camera module of an endoscope as claimed in claim 1, wherein the transparent ring being composed by an upper fringe, a middle fringe, and a lower fringe, wherein the outer diameter of the middle fringe is equal to the outer diameter of the tube and the cylinder body, the outer diameter of the upper fringe is equal to the inner diameter of the cylinder body, and the outer diameter of the lower fringe is equal to the inner diameter of the tube.

4. The improved lighting assembly for camera module of an endoscope as claimed in claim 1, wherein the LEDs emits the light through 360 degrees around the tube and cylinder body.
